(19) United States
(12) Patent Application Publication
(10) Pub. No.: US 2019/0008179 A1
(43) Pub. Date: Jan. 10, 2019

(54) COMPOSITION WITH REDUCED OFF-FLAVOR AND METHOD FOR PRODUCING SAME

(71) Applicant: AJINOMOTO CO., INC., Tokyo (JP)

(72) Inventors: Kota NAKAJO, Kawasaki-shi (JP); Susumu YAMAGUCHI, Kawasaki-shi (JP)

(73) Assignee: AJINOMOTO CO., INC., Tokyo (JP)

(21) Appl. No.: 16/130,199

(22) Filed: Sep. 13, 2018

Related U.S. Application Data
(63) Continuation of application No. PCT/JP2017/010312, filed on Mar. 15, 2017.

(30) Foreign Application Priority Data
Mar. 16, 2016 (JP) 2016-053092

(51) Int. Cl. A23D 9/007; A23L 27/00; A61K 47/44; A61K 47/18

(52) U.S. Cl. CPC A23D 9/007; A23L 27/86; A61K 47/44; A23V 2250/06; A23V 2002/00; A23V 2200/16; A61K 47/183

(57) ABSTRACT

Compositions containing (A) a substance with a foreign taste and (B) an oil or fat, wherein a content of (B) oil or fat is 3 to 400 parts by weight per 100 parts by weight of (A) substance with a foreign taste, and a difference between a solid fat content at 20° C. and a solid fat content at 40° C. of (B) oil or fat is not less than 11.5% are effective for suppressing the foreign taste of the substance with a foreign taste.

### CROSS REFERENCES TO RELATED APPLICATIONS

This application is a continuation of International Patent Application No. PCT/JP2017/010312, filed on Mar. 15, 2017, and claims priority to Japanese Patent Application No. 2016-053092, filed on Mar. 16, 2016, both of which are incorporated herein by reference in their entireties.

### BACKGROUND OF THE INVENTION

### Field of the Invention

The present invention relates to compositions with a suppressed foreign taste and production methods thereof. The present invention also relates to methods for suppressing a foreign taste.

### Discussion of the Background

As a method for masking a foreign taste such as bitter taste and the like, there have conventionally been reported, in the technical field of medicament, food and the like, methods for masking a foreign taste by adding oil or fat to a substance with a foreign taste. (e.g., bitter substance etc.) or by coating a substance with a foreign taste with oil or fat (see National Publication of International Patent Application No. 2014-518093, JP-A-2004-18829, WO 2009/122828, JP-A-2006-325542, and JP-A-2007-261985, all of which are incorporated herein by reference in their entireties). However, there is no report relating to the property of oils and fats suitable for reducing a foreign taste. There is neither a report relating to the property of oils and fats suitable for coating amino acid, powder, and the like.

On the other hand, several reports have been made relating to the property of oil or fat compositions used for coating, controlling the property of food and the like (see JP-A-2013-223466, JP-A-2012-244920, JP-A-2012-152113, JP-A-2011-103809, and J. Grad. Sch. Biosp. Sci., Hiroshima Univ. (2009), 48:77-94, all of which are incorporated herein by reference in their entireties). However, those oil or fat compositions are used for production of confectionery and bread such as bakery, chocolate, cream etc., for ice cream, for emulsified oil and the like requesting plasticity and extensibility of oils and fats.

Thus, there remains a need for compositions with a suppressed foreign taste and methods for suppressing a foreign taste.

### SUMMARY OF THE INVENTION

Accordingly, it is one object of the present invention to provide novel compositions which comprise a substance with a foreign taste and suppress the foreign taste.

It is another object of the present invention to provide novel methods for producing such a composition.

It is another object of the present invention to provide novel methods of suppressing a foreign taste.

These and other objects, which will become apparent during the following detailed description, have been achieved by the inventors' discovery that a foreign taste (e.g., bitter taste etc.) of a substance can be effectively suppressed by combining oil or fat having particular property that the difference between a solid fat content at 20° C. and a solid fat content at 40° C. is not less than 11.5% and the substance with a foreign taste (e.g., bitter substance etc.).

Accordingly, the present invention provides:

(1) A composition, comprising

(A) substance with a foreign taste; and

(B) oil or fat, wherein

a content of (B) oil or fat is 3 to 400 parts by weight per 100 parts by weight of (A) substance with a foreign taste, and

a difference between a solid fat content at 20° C. and a solid fat content at 40° C. of (B) oil or fat is not less than 11.5%.

(2) The composition of (1), wherein the foreign taste is a bitter taste.

(3) The composition of (1) or (2), wherein the substance with a foreign taste comprises amino acid.

(4) The composition of (3), wherein the amino acid comprises a branched chain amino acid.

(5) The composition of any one of (1) to (4) wherein (B) oil or fat has a solid fat content of 30 to 100% at 20° C.

(6) The composition of any one of (1) to (5) wherein (B) oil or fat has a solid fat content of 0 to 88.5% at 40° C.

(7) A medicament or food comprising the composition of any one of (1) to (6).

(8) A method for producing a composition, comprising combining (A) substance with a foreign taste and (B) oil or fat, wherein

a content of (B) oil or fat is 3 to 400 parts by weight per 100 parts by weight of (A) substance with a foreign taste, and

a difference between a solid fat content at 20° C. and a solid fat content at 40° C. of (B) oil or fat is not less than 11.5%.

(9) The method of (8), wherein the foreign taste is a bitter taste.

(10) The method of (8) or (9), wherein the substance with a foreign taste comprises amino acid.

(11) The method of (10), wherein the amino acid comprises a branched chain amino acid.

(12) The method of any one of (8) to (11) wherein (B) oil or fat has a solid fat content of 30 to 100% at 20° C.

(13) The method of any one of (8) to (12) wherein (B) oil or fat has a solid fat content of 0 to 88.5% at 40° C.

(14) A method for suppressing foreign taste of (A) substance with a foreign taste, comprising combining (A) substance with a foreign taste and (B) oil or fat, wherein

a content of (B) oil or fat is 3 to 400 parts by weight per 100 parts by weight of (A) substance with a foreign taste, and

a difference between a solid fat content at 20° C. and a solid fat content at 40° C. of (B) oil or fat is not less than 11.5%.

(15) The method of (14), wherein the foreign taste is a bitter taste.

(16) The method of (14) or (15), wherein the substance with a foreign taste comprises amino acid.

(17) The method of (16), wherein the amino acid comprises a branched chain amino acid.

(18) The method of any one of (14) to (17) wherein (B) oil or fat has a solid fat content of 30 to 100% at 20° C.

(19) The method of any one of (14) to (18) wherein (B) oil or fat has a solid fat content of 0 to 88.5% at 40° C.

### Effect of the Invention

According to the present invention, a composition comprising a substance with a foreign taste and suppressing the foreign taste, and a production method of the composition can be provided. According to the present invention, moreover, an effective method for suppressing a foreign taste can be provided.

### DETAILED DESCRIPTION OF THE PREFERRED EMBODIMENTS

### 1. Composition of the Present Invention

The composition of the present invention contains (A) a substance with a foreign taste (hereinafter to be also referred to as “Component A”) and (B) oil or fat (hereinafter to be also referred to as “Component B”).

Component a (Substance with a Foreign Taste)

A “substance with a foreign taste” to be used as Component A refers to a substance capable of producing a foreign taste in the mouth.

In the present invention, the “foreign taste” refers to an uncomfortable taste or flavor which is not felt by oral intake of a general food or medicament. Specific examples thereof include bitter taste, rough taste, pungent taste, harsh taste, astringent taste, foreign flavor and the like. Examples of the foreign flavor include uncomfortable flavor peculiar to amino acid, flavor such as putrefaction odor, flavor such as musty odor, fishy flavor, gamey flavor, oxidized odor, flavor such as solvents, disinfectant odor and the like. In addition, excessive sweetness, excessive sour taste, excessive umami and the like may also be a foreign taste. The presence or absence and the level of foreign taste can be evaluated by sensory evaluation by an expert panel as shown in the below-mentioned example.

Specific examples of a substance with a foreign taste include amino acids and salts thereof such as branched chain amino acid (valine, leucine and isoleucine), proline, phenylalanine, tyrosine, tryptophan, arginine, arginine hydrochloride, methionine, ornithine hydrochloride, histidine, cystine, cysteine and the like; alkaloids such as caffeine, theobromine, nicotine and the like; tannins such as catechin, tannin and the like; flavonoids such as naringin, hesperidin and the like; terpenoids such as limonin and the like; acetaminophen, ampicillin, azithromycin, phenylthiourea, quinine, strychnine and the like.

Examples of the kind of a substance with a foreign taste include bitter substance, rough taste substance, pungent taste substance, harsh taste substance, astringent taste substance, foreign flavor substance and the like.

Examples of the bitter substance include amino acids and salts thereof such as branched chain amino acid (valine, leucine and isoleucine), proline, phenylalanine, tyrosine, tryptophan, arginine, arginine hydrochloride, methionine, ornithine hydrochloride, histidine and the like; alkaloids such as caffeine, theobromine, nicotine and the like; tannins such as catechin, tannin and the like; flavonoids such as naringin, hesperidin and the like; terpenoids such as limonin and the like; acetaminophen, ampicillin, azithromycin, phenylthiourea, quinine, strychnine and the like.

Examples of the rough taste substance include epigallocatechin gallate, epicatechin gallate, leucodelphinidin-3-glycoside and the like.

Examples of the pungent taste substance include capsaicins, piperines, amides, isothiocyanates, sesqui terpenes, gingerols and the like.

Examples of the harsh taste substance include homogentisic acid, oxalic acid and the like.

Examples of the astringent taste substance include tannin and the like.

Examples of the foreign flavor substance include amino acids or salts thereof such as methionine and the like; substances capable of producing a putrefaction odor-like flavor such as ammonia, indole, skatole, hydrogen sulfide, volatility amine, mercaptan, fatty acid, butyric acid, valeric acid, putrescine, cadaverine, propionic acid and the like; substances capable of producing a musty odor-like flavor such as halogenated anisoles, 2-methylisoborneol, geosmin and the like; substances capable of producing a fishy flavor such as trimethylamine, 2,4-heptadienal and the like; substances capable of producing a gamey flavor such as cresol, isopropylphenol and the like; oxidized odor substances such as aldehydes (e.g., n-hexanal, 2-hexenal, 2,4-decadienal etc.) and the like; substances capable of producing a solvent-like flavor such as isopropyl alcohol, 1-butanol, methanol, xylene and the like; disinfectant odor substances such as ethanol, 2,4-dichlorophenol and the like; and the like.

Component A may be composed of one kind of substance with a foreign taste, or two or more kinds of substances with a foreign taste.

When Component A contains amino acid or consists of amino acid, the amino acid may be any of an L-form, a D-form and a DL-form. It is preferably an L-form or a DL-form, further preferably an L-form.

When Component A contains amino acid or consists of amino acid, the amino acid may be in the form of a salt. The salt of amino acid is not particularly limited as long as it has a foreign taste and is acceptable as a medicament or food. Examples thereof include salts with inorganic acids (e.g., hydrogen chloride, hydrogen bromide, sulfuric acid, phosphoric acid etc.); salts with organic acids (e.g., acetic acid, lactic acid, citric acid, tartaric acid, maleic acid, fumaric acid, monomethylsulfuric acid etc.); salts with inorganic bases (e.g., sodium, potassium, calcium, ammonia etc.); salts with organic bases (e.g., ethylenediamine, propylenediamine, ethanolamine, monoalkylethanolamine, dialkylethanolamine, diethanolamine, triethanolamine etc.) and the like. The salt of amino acid may be a hydrate (hydrate salt) and examples of the hydrate include 1 to 6 hydrates and the like.

When Component A contains three kinds of branched chain amino acids of isoleucine, leucine and valine or is composed of the branched chain amino acids, the weight ratio of isoleucine, leucine and valine (isoleucine:leucine:valine) is preferably 1:1 to 5:0.5 to 2.0, more preferably 1:1.5 to 3:0.8 to 1.7, particularly preferably 1:1.9 to 2.2:1.1 to 1.3.

The content of Component A in the composition of the present invention may be appropriately adjusted according to the kind of Component A and the like and is not particularly limited. In one embodiment, when Component A is a component desirably contained in the composition of the present invention for the use and effect expected of the composition of the present invention (e.g., when Component A is an active ingredient or an aid component of the effect expected of the composition of the present invention and the like), the content is preferably 50 to 95 wt %, more preferably 80 to 95 wt %, particularly preferably 85 to 95 wt %, of the composition of the present invention. Among the aforementioned Components A, for example, amino acid, caffeine and the like can be used as components desirably contained in the composition of the present invention. On the other hand, when Component A is not necessary for the composition of the present invention, the content of Component A is preferably as small as possible so that the development of a foreign taste can be suppressed as far as possible.

### Component B (Oil or Fat)

Component B preferably shows a particular range of difference between a solid fat content at 20° C. and a solid fat content at 40° C. By realizing coexistence of Component B showing a particular range of difference between a solid fat content at 20° C. and a solid fat content at 40° C. with Component A, the foreign taste of Component A can be effectively suppressed. The reason for the preference for the particular range of difference between a solid fat content at 20° C. and a solid fat content at 40° C. of Component B is not certain. However, since Component B has specific properties that it has a certain solid state at room temperature (e.g., 20° C.) and dissolves rapidly at mouth temperature (35° C. to 40° C.), it may have the effects of promoting dispersion of Component A (substance with a foreign taste) in the mouth and ejection thereof from the mouth during eating, or making it difficult to feel a foreign taste by an endothermic reaction upon dissolution.

To be specific, the difference between a solid fat content at 20° C. and a solid fat content at 40° C. of Component B is preferably not less than 11.5%, more preferably not less than 12%, still more preferably not less than 15%, further preferably not less than 20%, particularly preferably not less than 30%, most preferably not less than 35%. While the upper limit of the difference between a solid fat content at 20° C. and a solid fat content at 40° C. of Component B may be 100%, it is preferably 90%, more preferably 85%, particularly preferably 60%, because the foreign taste suppressing effect tends to be easily exhibited when Component B contains a solid fat to some extent at 40° C.

In the present invention, the “difference between a solid fat content at 20° C. and a solid fat content at 40° C. of oil or fat” refers to what is calculated by subtracting a solid fat content (%) of oil or fat at 40° C. from a solid fat content (%) of the oil or fat at 20° C. (i.e.:

(difference (%) between solid fat content at 20° C. and solid fat content at 40° C. of oil or fat)=(solid fat content (% by weight based on the total weight of Component B) of oil or fat at 20° C.)−(solid fat content (% by weight based on the total weight of Component B) of oil or fat at 40° C.)).

In the present invention, the solid fat content of oil or fat is measured according to “Japan Oil Chemists' Society; Standard Methods for the Analysis of Fats, Oils and Related Materials 2.2.9-2003 Solid Fat Content (NMR Method)” which is incorporated herein by reference in its entirety.

The solid fat content of Component B at 20° C. is preferably 30 to 100%, more preferably 35 to 97%, particularly preferably 40 to 90%.

The solid fat content of Component B at 40° C. is preferably 0 to 88.5%, more preferably 0 to 80%, still more preferably 0 to 60%, further preferably 10 to 60%, particularly preferably 20 to 60%, most preferably 30 to 60%.

It is preferable that Component B shows a difference between a solid fat content at 20° C. and a solid fat content at 40° C. of not less than 30% (preferably not less than 35%) and a solid fat content at 40° C. of not less than 10% (preferably not less than 20%) because a higher foreign taste suppressing effect is achieved.

The kind of Component B is not particularly limited as long as the difference between a solid fat content at 20° C. and a solid fat content at 40° C. falls within the aforementioned particular range. For example, oils and fats such as extremely hydrogenated palm oil, hydrogenated palm oil, palm oil, fractionated palm oil, extremely hydrogenated palm kernel oil, hydrogenated palm kernel oil, palm kernel oil, extremely hydrogenated coconut oil, hydrogenated coconut oil, coconut oil, extremely hydrogenated soybean oil, hydrogenated soybean oil, soybean oil, extremely hydrogenated rape seed oil, hydrogenated rape seed oil, rape seed oil, extremely hydrogenated olive oil, hydrogenated olive oil, olive oil, extremely hydrogenated almond oil, hydrogenated almond oil, almond oil, extremely hydrogenated avocado oil, hydrogenated avocado oil, avocado oil, extremely hydrogenated peanut oil, hydrogenated peanut oil, peanuts oil, extremely hydrogenated cottonseed oil, hydrogenated cottonseed oil, cottonseed oil, extremely hydrogenated corn oil, hydrogenated corn oil, corn oil, extremely hydrogenated safflower oil, hydrogenated safflower oil, safflower oil, extremely hydrogenated sunflower oil, hydrogenated sunflower oil, sunflower oil, extremely hydrogenated safflower oil, hydrogenated safflower oil, safflower oil, extremely hydrogenated rice oil, hydrogenated rice oil, rice oil and the like may be used singly or a mixture of two or more kinds thereof so that the difference between a solid fat content at 20° C. and a solid fat content at 40° C. falls within the aforementioned particular range. For example, when a mixture of extremely hydrogenated palm kernel oil and extremely hydrogenated palm oil is used, the weight ratio thereof (extremely hydrogenated palm kernel oil:extremely hydrogenated palm oil) is preferably 1:0.01 to 6, more preferably 1:0.3 to 3.

The slip melting point of Component B is preferably 20 to 60° C., more preferably 20 to 55° C., particularly preferably 25 to 55° C.

In the present invention, the slip melting point of oil or fat is measured according to “Japan Oil Chemists' Society; Standard Methods for the Analysis of Fats, Oils and Related Materials 2.2.4.2-1996 Melting Point (Slipping Point)” which is incorporated herein by reference in its entirety.

The content of Component B in the composition of the present invention may be appropriately adjusted according to the kind of Component A and the like and is not particularly limited. In one embodiment, when Component A is a component desirably contained in the composition of the present invention for the use and effect expected of the composition of the present invention, the content of Component B is preferably 3 to 50 wt %, more preferably 5 to 30 wt %, particularly preferably to 25 wt %, of the composition of the present invention, so that the ratio of Component A desirably contained in the composition of the present invention can be increased as much as possible while exhibiting the foreign taste suppressing effect by Component B.

While the total content of Component A and Component B in the composition of the present invention may be appropriately adjusted according to the kind and the like of Component A and is not particularly limited, in one embodiment where Component A is a component desirably contained in the composition of the present invention for the use and effect expected of the composition of the present invention, it is preferably 50 to 100 wt %, more preferably 80 to 100 wt %, particularly preferably 90 to 100 wt %, based on the total weight of the composition.

While the content of Component B in the composition of the present invention can be appropriately adjusted according to the intensity and the like of the foreign taste of Component A, it is preferably 3 to 400 parts by weight, more preferably 5 to 150 parts by weight, particularly preferably 10 to 100 parts by weight, per 100 parts by weight of Component A. When the content of Component B is less than 3 parts by weight per 100 parts by weight of Component A, the foreign taste tends to be felt strong. On the other hand, when the content of Component B exceeds 400 parts by weight per 100 parts by weight of Component A, Component B tends to affect the texture of the taken composition of the present invention. In addition, when Component B with a melting point of lower than 40° C. is used, the composition of the present invention tends to develop stickiness.

The composition of the present invention may further contain other components in addition to Component A and Component B. Such other components are not particularly limited as long as the object of the present invention is not impaired. Examples thereof include medicaments, raw material of medicaments, foods, foodstuffs, various efficacy components, excipients, suspending agents, dispersing agents, surfactants, thickeners, antioxidants, preservatives, pH adjusters, flavoring agents, colorants, sweeteners, proteins, carbohydrates, lipids, and the like. The content of said other components in the composition of the present invention can be appropriately adjusted according to the kind etc. of the other components and is not particularly limited. It is preferably not more than 50 wt %, more preferably not more than 30 wt %, particularly preferably not more than 20 wt % based on the total weight of the composition.

The production method of the composition of the present invention is not particularly limited except that Component A and Component B are combined. For example, it can be produced by appropriately combining the methods conventionally used in the field of medicament and food and a method analogous thereto as necessary and the like.

In the production method of the composition of the present invention, a method for combining Component A and Component B is not particularly limited as long as Component A and Component B coexist in the mouth when the composition of the present invention is taken. For example, a method including adding one of Component A and Component B to the other, and agitating the mixture as necessary by a powder handling apparatus, a speed kneader or the like, and the like can be mentioned.

In the production method of the composition of the present invention, Component A itself can be combined with Component B. Alternatively, a composition containing Component A and other substances (hereinafter to be also referred to as “Component A-containing material” for convenience) may be combined with Component B. The Component A-containing material may further contain, in addition to Component A, for example, medicament or a starting material of a medicament, food, foodstuff, various efficacy components, excipient, suspending agent, dispersing agent, surfactant, thickener, antioxidant, preservative, pH adjuster, flavoring agent, colorant and the like, though not limited thereto.

The content of Component A in the Component A-containing material may be appropriately adjusted according to the kind of Component A and the like and is not particularly limited. In one embodiment, when Component A is a component desirably contained in the composition of the present invention for the use and effect expected of the composition of the present invention (e.g., when Component A is an active ingredient or an aid component of the effect expected of the composition of the present invention and the like), the content is preferably 50 to 99.9 wt %, more preferably 70 to 99.9 wt %, particularly preferably 80 to 99.9 wt %, of the Component A-containing material. On the other hand, when Component A is not necessary for the composition of the present invention, the content of Component A is preferably as small as possible so that the development of a foreign taste can be suppressed as far as possible.

The content of the substance other than Component A in the Component A-containing material may be appropriately adjusted according to the kind of Component A, the kind of the substance other than Component A and the like and is not particularly limited. In one embodiment, when Component A is a component desirably contained in the composition of the present invention for the use and effect expected of the composition of the present invention, the content is preferably not more than 50 wt %, more preferably not more than 30 wt %, particularly preferably not more than 20 wt %, of the Component A-containing material.

The form of the Component A-containing material is not particularly limited and, for example, may be any of solid, liquid, semisolid (including jelly-like, slurry-like) and the like, with preference given to solid. To be specific, preferred are the forms of particulates (e.g., powder, fine particles, fine granules, granules, a mixture of at least two therefrom etc.), extruded molding, tabletted molding and the like. When the form of the Component A-containing material is solid, Component A may be partly or entirely coated with Component B. In combining with Component A, Component B may be melted by heating or may be combined in a state containing a solid without melting.

The production method of the Component A-containing material is not particularly limited. For example, it can be produced by appropriately combining the methods conventionally used in the field of medicament and food and a method analogous thereto as necessary and the like. For example, when the form of the Component A-containing material is a powder, the powder can be produced by subjecting Component A and a substance other than Component A to a treatment such as pulverization, mixing, granulation or the like as necessary and the like. A commercially available product may also be used as the Component A-containing material.

When the production method of the composition of the present invention includes combining a Component A-containing material and Component B, the amount of Component B is preferably 3 to 50 wt %, more preferably 5 to 30 wt %, particularly preferably 10 to 25 wt %, of the total amount of the Component A-containing material and Component B.

The composition of the present invention may be added to a medicament or food, or the composition of the present invention itself may be used as a medicament or food. Accordingly, the present invention also provides a medicament or food comprising the composition of the present invention. In the present invention, “food” is a concept widely encompassing those that can be taken orally and includes beverages and seasonings and the like.

The dosage form of a medicament containing the composition of the present invention is not particularly limited as long as it can be taken orally. Examples thereof include tablet, granule, powder, capsule (including soft capsule), elixir, syrup, microcapsule, drink, emulsion, suspension and the like, with preference given to tablet, granule and powder.

The form of a food containing the composition of the present invention is not particularly limited and, for example, may be any of solid, liquid, semisolid (including jelly-like, slurry-like) and the like, with preference given to solid. To be specific, preferred are the forms of particulates (e.g., powder, fine particles, fine granules, granules, a mixture of at least two therefrom etc.) and the like since a mixing state of Component A and Component B can be maintained with ease.

A food containing the composition of the present invention can be provided as, for example, food with health claims (food for specified health uses and food with nutrient function claims), dietary supplement (including nutritional supplementary food, health supplement and the like) or the like.

When the composition of the present invention is contained in a medicament or food, the content of the composition of the present invention in the medicament or food is generally 0.01 to 100 wt %, based on the total weight of the medicament or food.

The production method of a medicament or food containing the composition of the present invention is not particularly limited. For example, it can be produced by appropriately combining the methods conventionally used in the field of medicament and food and a method analogous thereto as necessary m and the like.

Examples of the target to which a medicament or food containing the composition of the present invention is administered or by which it is taken include mammals (e.g., human, mouse, rat, hamster, rabbit, cat, dog, bovine, sheep, monkey etc.) and the like. Preferred are humans.

The amount and frequency of administering or taking a medicament or food containing the composition of the present invention may be appropriately adjusted according to the components, form, and the like of the composition of the present invention. For example, when the composition of the present invention contains a branched chain amino acid, a food containing same can be taken such that the intake amount of the branched chain amino acid per meal for an adult is 0.5 to 3 g.

### 2. Foreign Taste Suppressing Method of the Present Invention

The foreign taste suppressing method of the present invention includes combining Component A and Component B. Component A and Component B used in the foreign taste suppressing method of the present invention are the same as those contained in the composition of the present invention, and preferable embodiments thereof are also the same.

The amount of Component B is preferably 3 to 400 parts by weight, more preferably 5 to 150 parts by weight, particularly preferably 10 to 100 parts by weight, per 100 parts by weight of Component A. When the amount of Component B is less than 3 parts by weight per 100 parts by weight of Component A, the foreign taste tends to be felt strong. On the other hand, when the amount of Component B exceeds 400 parts by weight per 100 parts by weight of Component A, Component B tends to affect the texture of the taken composition of the present invention. In addition, when Component B with a melting point of lower than 40° C. is used, the composition of the present invention tends to develop stickiness.

In the foreign taste suppressing method of the present invention, a method for combining Component A and Component B is not particularly limited as long as Component A and Component B coexist in the mouth when they are taken. For example, yielding a single composition of Component A and Component B (e.g., the composition of the present invention etc.) by a method including adding one of Component A and Component B to the other, and agitating the mixture as necessary by a powder handling apparatus, a speed kneader or the like, and the like can be mentioned. These components may be coexisted in the mouth by placing either Component A or Component B in the mouth and then placing the other in the mouth.

In the foreign taste suppressing method of the present invention, Component A itself can be combined with Component B. Alternatively, a Component A-containing material may be combined with Component B. The Component A-containing material usable in the foreign taste suppressing method of the present invention is the same as that usable in the aforementioned production method of the composition of the present invention, and preferable embodiments thereof are also the same.

When the foreign taste suppressing method of the present invention includes combining a Component A-containing material and Component B, the amount of Component B is preferably 3 to 50 wt %, more preferably 5 to 30 wt %, particularly preferably 10 to 25 wt %, of the total amount of the Component A-containing material and Component B.

According to the foreign taste suppressing method of the present invention, the foreign tastes of Component A (e.g., bitter taste, rough taste, pungent taste, harsh taste, foreign flavor (particularly uncomfortable flavor peculiar to amino acid), excessive sweetness, excessive sour taste, excessive umami etc.) can be suppressed.

Other features of the invention will become apparent in the course of the following descriptions of exemplary embodiments which are given for illustration of the invention and are not intended to be limiting thereof.

### EXAMPLES

Preparation of Material Containing Substance with Foreign Taste

Respective amino acids were mixed and pulverized at the compositions (wt %) shown in Table 1 to prepare powdery amino acid mixtures 1 and 2.

TABLE 1

| amino acid | | | composition (wt %) | |
|---|---|---|---|---|
| | | foreign taste | amino | amino |
| | bitter | other than | acid | acid |
| component name | taste | bitter taste | mixture 1 | mixture 2 |
| L-leucine | present | — | 50 | 40.0 |
| L-lysine | — | — | — | 16.7 |
| hydrochloride | | | | |
| L-valine | present | — | 25 | 11.0 |
| L-isoleucine | present | — | 25 | 10.6 |
| L-threonine | — | — | — | 9.3 |
| L-phenylalanine | present | — | — | 6.7 |
| L-methionine | — | present | — | 3.3 |
| L-histidine | — | — | — | 1.7 |
| hydrochloride | | | | |
| L-tryptophan | present | — | — | 0.7 |

| | | |
|---|---|---|
| bitter substance in total | 100 | 69.0 |
| total substances with foreign taste other | 0 | 3.3 |
| than bitter substance | | |
| total all substances with foreign taste | 100 | 72.3 |
| (bitter substance + substance with foreign | | |
| taste other than bitter substance) | | |

### Preparation of Oil or Fat

Extremely hydrogenated palm oil and extremely hydrogenated palm kernel oil were mixed at the weight ratios shown in Table 2 to prepare oil or fat 3 to 10.

In addition, extremely hydrogenated rape seed oil, extremely hydrogenated palm oil, extremely hydrogenated palm kernel oil, coconut oil, red palm oil (palm olein) and rape seed oil were respectively used as oil or fat 1, 2, and 11 to 14.

TABLE 2

| | |
|---|---|
| oil or fat 1 | extremely hydrogenated rape seed oil |
| oil or fat 2 | extremely hydrogenated palm oil |
| oil or fat 3 | mixture of extremely hydrogenated palm oil and |
| | extremely hydrogenated palm kernel oil |
| | (extremely hydrogenated palm oil:extremely |
| | hydrogenated palm kernel oil = 70:1) |
| oil or fat 4 | mixture of extremely hydrogenated palm oil and |
| | extremely hydrogenated palm kernel oil |
| | (extremely hydrogenated palm oil:extremely |
| | hydrogenated palm kernel oil = 8:1) |
| oil or fat 5 | mixture of extremely hydrogenated palm oil and |
| | extremely hydrogenated palm kernel oil |
| | (extremely hydrogenated palm oil:extremely |
| | hydrogenated palm kernel oil = 6:1) |
| oil or fat 6 | mixture of extremely hydrogenated palm oil and |
| | extremely hydrogenated palm kernel oil |
| | (extremely hydrogenated palm oil:extremely |
| | hydrogenated palm kernel oil = 3:1) |
| oil or fat 7 | mixture of extremely hydrogenated palm oil and |
| | extremely hydrogenated palm kernel oil |
| | (extremely hydrogenated palm oil:extremely |
| | hydrogenated palm kernel oil = 2:1) |
| oil or fat 8 | mixture of extremely hydrogenated palm oil and |
| | extremely hydrogenated palm kernel oil |
| | (extremely hydrogenated palm oil:extremely |
| | hydrogenated palm kernel oil = 1:1) |
| oil or fat 9 | mixture of extremely hydrogenated palm oil and |
| | extremely hydrogenated palm kernel oil |
| | (extremely hydrogenated palm oil:extremely |
| | hydrogenated palm kernel oil = 1:2) |
| oil or fat 10 | mixture of extremely hydrogenated palm oil and |
| | extremely hydrogenated palm kernel oil |
| | (extremely hydrogenated palm oil:extremely |
| | hydrogenated palm kernel oil = 1:3) |
| oil or fat 11 | extremely hydrogenated palm kernel oil |
| oil or fat 12 | coconut oil |
| oil or fat 13 | red palm oil (palm olein) |
| oil or fat 14 | rape seed oil |

### Measurement of Solid Fat Content

The solid fat content (%) of oil or fat 1 to 14 at 10 to 60° C. was measured according to “Japan Oil Chemists' Society; Standard Methods for the Analysis of Fats, Oils and Related Materials 2.2.9-2003 Solid Fat Content (NMR Method)” which is incorporated herein by reference in its entirety.

### Measurement of Slip Melting Point

The slip melting point (° C.) of oil or fat 1 to 14 was measured according to “Japan Oil Chemists' Society; Standard Methods for the Analysis of Fats, Oils and Related Materials 2.2.4.2-1996 Melting Point (Slipping Point)” which is incorporated herein by reference in its entirety.

The results are shown in Table 3-1 and Table 3-2.

TABLE 3-1

| | measurement | oil or fat | | | | | | |
|---|---|---|---|---|---|---|---|---|
| | temperature (° C.) | 1 | 2 | 3 | 4 | 5 | 6 | 7 |
| solid fat | 10 | about 100 | 98.3 | 98.5 | 97.7 | 97.5 | 96.5 | 96.4 |
| content | 20 | about 100 | 97.9 | 101.8 | 100.1 | 99.6 | 95.4 | 96.5 |
| (%) | 30 | about 100 | 97.6 | 96.9 | 93.7 | 92.5 | 89.1 | 84.0 |
| | 35 | about 100 | 97.5 | 97.1 | 92.1 | 90.3 | 84.1 | 78.1 |
| | 40 | about 100 | 97.5 | 96.4 | 89.6 | 87.3 | 79.6 | 72.4 |
| | 45 | about 100 | 96.2 | 94.9 | 85.9 | 83.0 | 73.4 | 64.9 |
| | 50 | — | 94.2 | 92.4 | 79.0 | 74.8 | 61.9 | 51.2 |
| | 55 | — | 80.0 | 76.6 | 54.7 | 48.3 | 29.1 | 18.0 |
| | 60 | — | 2.0 | 1.7 | 1.0 | 0.8 | 0.2 | 0 |

| | | | | | | | |
|---|---|---|---|---|---|---|---|
| difference (%) between | about 0 | 0.5 | 5.4 | 10.5 | 12.2 | 15.8 | 24.0 |
| solid fat content at | | | | | | | |
| 20° C. and solid fat | | | | | | | |
| content at 40° C. | | | | | | | |
| slip melting point (° C.) | 68.0 | 58.0 | not | not | not | not | not |
| | | | measured | measured | measured | measured | measured |

TABLE 3-2

| | measurement | oil or fat | | | | | | |
|---|---|---|---|---|---|---|---|---|
| | temperature (° C.) | 8 | 9 | 10 | 11 | 12 | 13 | 14 |
| solid fat | 10 | 95.8 | 95.4 | 95.4 | 95.2 | 80.6 | 28.0 | 0 |
| content | 20 | 92.9 | 91.3 | 89.8 | 88.5 | 39.0 | 3.0 | 0 |
| (%) | 30 | 73.7 | 63.0 | 56.9 | 33.9 | 0.2 | 3.0 | 0 |
| | 35 | 64.7 | 50.2 | 42.0 | 13.9 | 0 | 0 | 0 |
| | 40 | 57.6 | 41.8 | 33.3 | 7.0 | 0.4 | 0 | 0 |
| | 45 | 48.2 | 31.7 | 23.2 | 2.2 | 0 | 0 | 0 |
| | 50 | 32.1 | 16.4 | 9.3 | 0.3 | — | 0 | 0 |
| | 55 | 3.1 | 1.5 | 0.2 | 0 | — | 0 | 0 |
| | 60 | 0 | 0 | 0.3 | 0.3 | — | 0 | 0 |

| | | | | | | | |
|---|---|---|---|---|---|---|---|
| difference (%) between | 35.3 | 49.6 | 56.5 | 81.5 | 38.6 | 3.0 | 0 |
| solid fat content at | | | | | | | |
| 20° C. and solid fat | | | | | | | |
| content at 40° C. | | | | | | | |
| slip melting point (° C.) | 54.2 | not | 50.6 | 41.5 | 24.4 | 19.0 | — |
| | | measured | | | | | |

### Experimental Example 1

### Preparation of Mixtures of Examples 1 to 8 and Comparative Examples 1 to 6

To oil or fat 1 to 14 (68 g) melted using ALL-PURPOSE POWDER HANDLING APPARATUS (FOR LAB) (manufactured by OKADA SEIKO Co., Ltd., MECHANOMill NEW MM-20) was gradually added amino acid mixture 1 (332 g) and the mixtures were stirred to give the mixtures of Examples 1 to 8 and Comparative Examples 1 to 6. Stirring was performed at 200 to 900 rpm while observing the state of the mixtures and the mixtures were uniformized.

### Sensory Evaluation

Two professional panelists took the mixtures (0.5 g as amino acid mixture) of Examples 1 to 8 and Comparative Examples 1 to 6, scored the intensity of the bitter taste felt at that time and the average point was taken as the bitter taste intensity. Scoring was performed with the bitter taste of the amino acid mixture itself (control) without addition of oil or fat as 10 points, and 1 point when a bitter taste was hardly felt.

The bitter taste suppressing effect of Examples 1 to 8 and Comparative Examples 1 to 6 was judged based on the following criteria.

### Diagnostic Criteria

x: no bitter taste suppressing effect (bitter taste intensity not more than 10 points to not less than 7.5 points)

Δ: bitter taste suppressing effect was present (bitter taste intensity less than 7.5 points to not less than 4.5 points)

◯: high bitter taste suppressing effect was present (bitter taste intensity less than 4.5 points to not less than 3.0 points)

⊙: very high bitter taste suppressing effect was present (bitter taste intensity less than 3.0 points to not less than 2.0 points)

⊙⊙: extremely high bitter taste suppressing effect was present (bitter taste intensity less than 2.0 points to not less than 1.0 point)

The results are shown in Table 4-1 and Table 4-2.

TABLE 4-1

| | Comparative | Comparative | Comparative | Comparative | | | |
|---|---|---|---|---|---|---|---|
| | Example 1 | Example 2 | Example 3 | Example 4 | Example 1 | Example 2 | Example 3 |
| sample | amino acid mixture 1 | | | | | | |
| (B) oil or fat | oil or | oil or | oil or | oil or | oil or | oil or | oil or |
| | fat 1 | fat 2 | fat 3 | fat 4 | fat 5 | fat 6 | fat 7 |
| amount (parts by weight) of (B) oil | 20.5 | 20.5 | 20.5 | 20.5 | 20.5 | 20.5 | 20.5 |
| or fat per 100 parts by weight of | | | | | | | |
| (A) substance with foreign taste | | | | | | | |
| bitter taste intensity (points) | 6 | 5 | 4.5 | 4.5 | 4 | 2.5 | 2 |
| judgment of bitter taste suppression | Δ | Δ | Δ | Δ | ◯ | ⊙ | ⊙ |

TABLE 4-2

| | | | | | | Comparative | Comparative |
|---|---|---|---|---|---|---|---|
| | Example 4 | Example 5 | Example 6 | Example 7 | Example 8 | Example 5 | Example 6 |
| sample | amino acid mixture 1 | | | | | | |
| (B) oil or fat | oil or | oil or | oil or | oil or | oil or | oil or | oil or |
| | fat 8 | fat 9 | fat 10 | fat 11 | fat 12 | fat 13 | fat 14 |
| amount (parts by weight) of (B) oil | 20.5 | 20.5 | 20.5 | 20.5 | 20.5 | 20.5 | 20.5 |
| or fat per 100 parts by weight of | | | | | | | |
| (A) substance with foreign taste | | | | | | | |
| bitter taste intensity (points) | 1.5 | 1.5 | 1 | 2 | 2 | 4.5 | 4.5 |
| judgment of bitter taste suppression | ⊙⊙ | ⊙⊙ | ⊙⊙ | ⊙ | ⊙ | Δ | Δ |

As is clear from the results shown in Table 4-1 and Table 4-2, a high bitter taste suppressing effect was confirmed in Example 1 in which the difference between a solid fat content at 20° C. and a solid fat content at 40° C. of oil or fat (Component B) was 12.2%, a very high bitter taste suppressing effect was confirmed in Examples 2, 3, 7, and 8 in which the difference was 15.8%, 24.0%, 81.5% or 38.6%, and an extremely high bitter taste suppressing effect was confirmed in Examples 4 to 6 in which the difference was 35.3%, 49.6% or 56.5%. In Example 4, a higher bitter taste suppressing effect was confirmed even though the difference between a solid fat content at 20° C. and a solid fat content at 40° C. of Component B was smaller than Examples 7 and 8. Thus, it is considered that the bitter taste suppressing effect is high in oil or fat in which some solid fat remains at 40° C. (e.g., oil or fat with a solid fat content at 40° C. of not less than 10% etc.).

### Experimental Example 2

### Preparation of Mixtures of Examples 9 to 12 and Comparative Example 7

To oil or fat 1 and 10 to 12 melted using a speed kneader (manufactured by OKADA SEIKO Co., Ltd., NSK-150S type) was gradually added amino acid mixture 2 at the mixing ratios shown in Table 5 to a total 400 g and the mixtures were stirred to give the mixtures of Examples 9 to 12 and Comparative Example 7. Stirring was performed at 300 to 1500 rpm while observing the state of the mixtures and the mixtures were uniformized.

### Sensory Evaluation

Two professional panelists took the mixtures (0.5 g as amino acid mixture) of Examples 9 to 12 and Comparative Example 7, scored the intensity of the bitter taste felt at that time and the intensity of the foreign flavor derived from amino acid (particularly methionine) and the average points were taken as the bitter taste intensity and foreign flavor intensity. Scoring of the bitter taste was performed in the same manner as in Experimental Example 1 and the scoring of the foreign flavor was performed with the foreign flavor of the amino acid mixture itself (control) without addition of oil or fat as 10 points, and 1 point when a foreign flavor was hardly felt.

In addition, the bitter taste suppressing effect in Examples 9 to 12 and Comparative Example 7 was judged based on the criteria similar to those in Experimental Example 1, and the foreign flavor suppressing effect in Examples 9 to 12 and Comparative Example 7 was judged based on the following criteria.

### Diagnostic Criteria

x: no foreign flavor suppressing effect (foreign flavor intensity not more than 10 points to not less than 7.5 points)

Δ: foreign flavor suppressing effect was present (foreign flavor intensity less than 7.5 points to not less than 4.5 points)

◯: high foreign flavor suppressing effect was present (foreign flavor intensity less than 4.5 points to not less than 3.0 points)

⊙: very high foreign flavor suppressing effect was present (foreign flavor intensity less than 3.0 points to not less than 2.0 points)

⊙⊙: extremely high foreign flavor suppressing effect was present (foreign flavor intensity less than 2.0 points to not less than 1.0 point)

The results are shown in Table 5.

TABLE 5

| | Comparative | | | | | |
|---|---|---|---|---|---|---|
| | Example 7 | Example 9 | Example 10 | Example 11 | Example 12 | control |
| sample | amino acid mixture 2 | | | | | |
| sample amount (parts by weight) | 80 | 80 | 80 | 83 | 83 | — |
| amount (parts by weight) of (A) substance | 57.9 | 57.9 | 57.9 | 60.0 | 60.0 | — |
| with foreign taste | | | | | | |
| (B) oil or fat | oil or | oil or | oil or | oil or | oil or | no |
| | fat 1 | fat 10 | fat 11 | fat 10 | fat 12 | addition |
| amount (parts by weight) of (B) oil | 20 | 20 | 20 | 17 | 17 | — |
| or fat | | | | | | |
| amount (parts by weight) of (B) oil | 34.6 | 34.6 | 34.6 | 28.3 | 28.3 | — |
| or fat per 100 parts by weight of | | | | | | |
| (A) substance with foreign taste | | | | | | |
| bitter taste intensity (points) | 5 | 2.5 | 2.5 | 1 | 2 | 10 |
| judgment of bitter taste suppression | Δ | ⊙ | ⊙ | ⊙⊙ | ⊙ | X |
| foreign flavor intensity (points) | 5 | 2 | 2 | 1 | 2 | 10 |
| judgment of foreign flavor suppression | Δ | ⊙ | ⊙ | ⊙⊙ | ⊙ | X |

As is clear from the results shown in Table 5, a very high bitter taste suppressing effect and foreign flavor suppressing effect were confirmed in Examples 9, 10, and 12 in which the difference between a solid fat content at 20° C. and a solid fat content at 40° C. of oil or fat (Component B) was 56.5%, 81.5%, or 38.6%, and extremely high bitter taste suppressing effect and foreign flavor suppressing effect were confirmed in Example 11 in which the difference was 56.5% and the amount of Component B was 28.3 parts by weight per 100 parts by weight of the substance with a foreign taste (Component A).

### Experimental Example 3

### Preparation of Mixtures of Examples 13 to 19 and Comparative Examples 8 to 12

To oil or fat 1 and 10 melted using ALL-PURPOSE POWDER HANDLING APPARATUS (FOR LAB) (manufactured by OKADA SEIKO Co., Ltd., MECHANOMill NEW MM-20) was gradually added amino acid mixture 1 at the mixing ratios shown in Table 6-1 or Table 6-2 to a total 400 g and the mixtures were stirred to give the mixtures of Examples 13 to 19 and Comparative Examples 8 to 12. Stirring was performed at 200 to 900 rpm while observing the state of the mixtures and the mixtures were uniformized.

### Sensory Evaluation

Two professional panelists took the mixtures (0.5 g as amino acid mixture) of Examples 13 to 19 and Comparative Examples 8 to 12, scored the bitter taste felt at that time and the average point was taken as the bitter taste intensity. Scoring was performed in the same manner as in Experimental Example 1.

In addition, the bitter taste suppressing effect in Examples 13 to 19 and Comparative Examples 8 to 12 was judged based on the criteria similar to those in Experimental Example 1.

The results are shown in Table 6-1 and Table 6-2.

TABLE 6-1

| | | Comparative | | | | | |
|---|---|---|---|---|---|---|---|
| | control | Example 8 | Example 13 | Example 14 | Example 15 | Example 16 | Example 17 |
| sample | amino acid mixture 1 | | | | | | |
| (B) oil or fat | no | oil or | oil or | oil or | oil or | oil or | oil or |
| | addition | fat 10 | fat 10 | fat 10 | fat 10 | fat 10 | fat 10 |
| amount (parts by weight) of (B) oil | — | 1.5 | 3.1 | 5.3 | 11.1 | 20.5 | 33.3 |
| or fat per 100 parts by weight of | | | | | | | |
| (A) substance with foreign taste | | | | | | | |
| bitter taste intensity (points) | 10 | 4.5 | 3.5 | 2 | 1.5 | 1 | 1 |
| judgment of bitter taste suppression | X | Δ | ◯ | ⊙ | ⊙⊙ | ⊙⊙ | ⊙⊙ |

TABLE 6-2

| | | | Comparative | Comparative | Comparative | Comparative |
|---|---|---|---|---|---|---|
| | Example 18 | Example 19 | Example 9 | Example 10 | Example 11 | Example 12 |
| sample | amino acid mixture 1 | | | | | |
| (B) oil or fat | oil or | oil or | oil or | oil or | oil or | oil or |
| | fat 10 | fat 10 | fat 1 | fat 1 | fat 1 | fat 1 |
| amount (parts by weight) of (B) oil | 42.9 | 100.0 | 1.5 | 3.1 | 5.3 | 20.5 |
| or fat per 100 parts by weight of | | | | | | |
| (A) substance with foreign taste | | | | | | |
| bitter taste intensity (points) | 1 | 1 | 10 | 8.5 | 8 | 6 |
| judgment of bitter taste suppression | ⊙⊙ | ⊙⊙ | X | X | X | Δ |

As is clear from the results shown in Table 6-1 and Table 6-2, a high bitter taste suppressing effect was confirmed in Example 13 in which the difference between a solid fat content at 20° C. and a solid fat content at 40° C. of oil or fat (Component B) was 56.5% and the amount of Component B was 3.1 parts by weight per 100 parts by weight of the substance with a foreign taste (Component A), a very high bitter taste suppressing effect was confirmed in Example 14 in which the amount of Component B was 5.3 parts by weight per 100 parts by weight of Component A, and an extremely high bitter taste suppressing effect was confirmed in Examples 15 to 19 in which the amount of Component B was 11.1 parts by weight, 20.5 parts by weight, 33.3 parts by weight, 42.9 parts by weight or 100.0 parts by weight, per 100 parts by weight of Component A. In Comparative Example 8 in which the difference between a solid fat content at 20° C. and a solid fat content at 40° C. of Component B was 56.5% but the amount of Component B was 1.5 parts by weight per 100 parts by weight of the substance with a foreign taste (Component A), a high bitter taste suppressing effect was not continued.

### Experimental Example 4

### Preparation of Mixtures of Example 20 and Comparative Example 13

To oil or fat 1 and 10 melted using ALL-PURPOSE POWDER HANDLING APPARATUS (FOR LAB) (manufactured by OKADA SEIKO Co., Ltd., MECHANOMill NEW MM-20) was gradually added a caffeine-containing powder (mixture of dextrin (3 parts by weight) per 1 part by weight of caffeine) at the mixing ratios shown in Table 7 to a total 400 g and the mixtures were stirred to give the mixtures of Example 20 and Comparative Example 13. Stirring was performed at 200 to 900 rpm while observing the state of the mixtures and the mixtures were uniformized.

### Sensory Evaluation

Two professional panelists took the mixtures (0.125 g as caffeine) of Example 20 and Comparative Example 13, scored the bitter taste felt at that time and the average point was taken as the bitter taste intensity. Scoring was performed with the bitter taste of the caffeine-containing powder itself (control) without addition of oil or fat as 10 points, and 1 point when the bitter taste was hardly felt.

In addition, the bitter taste suppressing effect in Example 20 and Comparative Example 13 was judged based on the criteria similar to those in Experimental Example 1.

The results are shown in Table 7.

TABLE 7

| | | Comparative | |
|---|---|---|---|
| | Example 20 | Example 13 | control |
| (A) substance with foreign | caffeine | | |
| taste | | | |
| (B) oil or fat | oil or fat | oil or fat 1 | no |
| | 10 | | addition |
| amount (parts by weight) | 81.9 | 81.9 | — |
| of (B) oil or fat per 100 | | | |
| parts by weight of (A) | | | |
| substance with foreign | | | |
| taste | | | |
| bitter taste intensity | 2 | 7.5 | 10 |
| (points) | | | |
| judgment of bitter taste | ⊙ | x | x |
| suppression | | | |

As is clear from the results shown in Table 7, a high caffeine bitter taste suppressing effect was confirmed in Example 20 in which the difference between a solid fat content at 20° C. and a solid fat content at 40° C. of oil or fat (Component B) was 56.5%.

### INDUSTRIAL APPLICABILITY

According to the present invention, a composition comprising a substance with a foreign taste and suppressing the foreign taste and a production method of the composition can be provided. According to the present invention, moreover, an effective method for suppressing a foreign taste can be provided.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition, comprising
(A) at least one substance with a foreign taste; and
(B) an oil or fat, wherein
a content of said (B) oil or fat is 3 to 400 parts by weight per 100 parts by weight of said (A) substance with a foreign taste, and
a difference between a solid fat content at 20° C. and a solid fat content at 40° C. of said (B) oil or fat is not less than 11.5%.

2. The composition according to claim 1, wherein said foreign taste is a bitter taste.

3. The composition according to claim 1, wherein said substance with a foreign taste comprises at least one amino acid.

4. The composition according to claim 3, wherein said at least one amino acid comprises a branched chain amino acid.

5. The composition according to claim 1, wherein said (B) oil or fat has a solid fat content of 30 to 100% at 20° C.

6. The composition according to claim 1, wherein said (B) oil or fat has a solid fat content of 0 to 88.5% at 40° C.

7. A medicament or food, comprising a composition according to claim 1.

8. A method for producing a composition, said method comprising combining (A) at least one substance with a foreign taste and (B) an oil or fat, wherein
said (B) oil or fat is combined with said (A) at least one substance with a foreign taste in an amount of 3 to 400 parts by weight of said (B) oil or fat per 100 parts by weight of said (A) substance with a foreign taste, and
a difference between a solid fat content at 20° C. and a solid fat content at 40° C. of said (B) oil or fat is not less than 11.5%.

9. A method for suppressing a foreign taste of (A) a substance with a foreign taste, comprising combining said (A) substance with a foreign taste and (B) an oil or fat, wherein
said (B) oil or fat is combined with said (A) substance with a foreign taste in an amount of 3 to 400 parts by weight of said (B) oil or fat per 100 parts by weight of said (A) substance with a foreign taste, and
a difference between a solid fat content at 20° C. and a solid fat content at 40° C. of said (B) oil or fat is not less than 11.5%.

10. The method according to claim 9, wherein said foreign taste is a bitter taste.

11. The method according to claim 9, wherein said substance with a foreign taste comprises at least one amino acid.

12. The method according to claim 11, wherein said at least one amino acid comprises a branched chain amino acid.

13. The method according to claim 9, wherein said (B) oil or fat has a solid fat content of 30 to 100% at 20° C.

14. The method according to claim 9, wherein said (B) oil or fat has a solid fat content of 0 to 88.5% at 40° C.
